(19) United States
(12) Patent Application Publication
(10) Pub. No.: US 2007/0137647 A1
(43) Pub. Date: Jun. 21, 2007

(54) Expiratory valve unit

(76) Inventor: Ian Dampney, London (GB)

Correspondence Address: HODGSON RUSS LLP, ONE M & T PLAZA, SUITE 2000, BUFFALO, NY, 14203-2391, US

(21) Appl. No.: 10/556,958

(22) PCT Filed: May 17, 2004
(86) PCT No.: PCT/GB04/02144

§ 371 Date: Aug. 15, 2006

(30) Foreign Application Priority Data
May 16, 2003 (GB) 0311338.8

(51) Int. Cl. A61M 16/00; A62B 18/02

(52) U.S. Cl. 128/204.18; 128/203.11; 128/205.24; 128/206.21

(57) ABSTRACT

An expiratory valve unit comprises a body having a plurality of outlets and valve seats, and a rotary closure member having a plurality of blades each normally biassed against a respective seat to close the outlets and being rotatable relative to said body when under pressure from user exhalate impinging the blades to open the outlets.

### BACKGROUND OF THE INVENTION

The present invention relates to an expiratory valve unit, and to a respirator incorporating the same. The invention also relates to a kit of parts for fitting breathing apparatus to the face of a wearer.

In modern combat aircraft breathing gas is supplied to a face mask worn by the airman. The breathing gas is pressurized and is drawn into the mask through a non-return, inspiratory valve therein as the airman inhales. When the airman exhales an expiratory valve allows the exhalate to be vented to the atmosphere. It is essential that this second valve only opens when the airman exhales. However, conventional expiratory valves are not suitable for service in high agility aircraft, because during maneuvers which generate high G forces the valves are either forced open, allowing breathing gas to vent directly from the supply to the atmosphere, or closed, making it impossible for the airman to exhale. For example, a known type of poppet valve has a spring loaded disc moving on the axis of a cylinder and is inherently sensitive to any acceleration along that axis.

It is an aim of at least the preferred embodiment of the present invention to solve these and other problems.

### SUMMARY OF THE INVENTION

In one aspect the present invention provides an expiratory valve unit comprising a body having an outlet, and a rotary closure member mounted for rotation about an axis passing through the centre of gravity of the closure member, the closure member being normally biassed against a valve seat to close the outlet and rotatable relative to said body when under pressure from user exhalate to open the outlet. This can provide a balanced system that can deliver use substantially independent from any G forces acting thereon.

In another aspect the present invention provides an expiratory valve unit comprising a body having a plurality of outlets, and a rotary closure member having a plurality of blades, the closure member being normally biassed against a valve seat to close the outlets and rotatable relative to said body when pressure from user exhalate acts on the blades to open the outlets.

Preferably the blades are symmetrically disposed about the closure member. The or each blade is preferably helical.

The axis of rotation of the closure member preferably extends in the direction of exhalate flow through the or each outlet. Preferably, the axis of rotation of the closure member extends substantially parallel to the direction of exhalate flow through the or each outlet.

There may be means for equalizing pressure differences across the closure member which would affect rotation thereof, other than pressure differences due to exhalate pressure. Preferably the unit comprises damping means for damping movement of the closure member. The damping means may comprise a part closely fitting and moveable in a closed chamber. Such part may comprise surfaces extending radially and preferably intersecting the axis of rotation of the closure member.

The pressure compensating means may be arranged for equalizing the pressure between a user side of the unit and the closed chamber when the user is not exhaling. The pressure compensating means may comprise gas inlets on the body in communication with the closed chamber for providing gas at user side pressure thereto.

The invention extends to a respirator comprising one of the valve units of the preceding aspects. Respirators for providing breathing gas to a crew member are typically designed to cover the wearer's nose and mouth, and include a rigid shell with fastenings to attach it to the wearer's head and into which passes a feed tube for the breathing gas. A flexible face seal, typically moulded from sheet rubber, is enclosed in the shell. The breathing apparatus, including the inspiratory and expiratory valves, together with a communications microphone, are housed in the flexible face seal.

Existing data about facial characteristics refers primarily to surface features and their dimensions. However, the tissues of the face have varying degrees of compliance, with the compliant areas being supported on a rigid but articulated infrastructure. Therefore, the size of the rubber seal must be sufficient to accommodate for such variations in the compliance of the facial tissues, as well as for variations in face shapes and sizes between crew members. However, increased use of such high density/low structural strength materials leads to an undesirable increase the weight of the mask.

It is another aim of the preferred embodiment of the present invention to solve these and other problems.

In another aspect, the present invention provides a kit of parts for connecting breathing apparatus to the face of a wearer, the kit comprising a range of similar pre-formed units of varying shape and/or size for selective connection, according to the shape and/or size of the wearer's head, to a second unit housing said breathing apparatus, each pre-formed unit having a flexible body for forming a seal with a wearer's face.

By providing a range of pre-formed units in various sizes, the units can be readily selected to custom-fit the breathing apparatus to an individual airman. Optical scanning using a laser or the like can provide an accurate profile of the wearer's face to enable the most suitable pre-formed unit to be selected for use by that airman. This can enable a reduction in the amount of rubber material used, whilst still enabling an effective seal to be formed with the wearer's face during use. Replacement of the redundant rubber material with a lighter, more rigid material, such as plastics material, can allow for overall mask weight reduction as well as increasing the strength of the mask. Use of precision tooling can allow accurate control of component geometry so that the mass of the unit is minimized.

Each pre-formed unit preferably comprises means for preventing inversion of the sealing surface of the unit when under pressure. The means may be integral with the seal.

The kit preferably comprises a range of similar pre-formed rigid units of varying shape each for selectively connecting a respective first pre-formed unit to the second unit.

This aspect of the present invention also extends to a method of connecting breathing apparatus to the face of a wearer, comprising connecting to a unit housing said breathing apparatus a second unit having a flexible body for forming a seal with a wearer's face, the second unit having been selected from a range of similar units of varying shape and/or size according to the shape and/or size of the wearer's face.

### BRIEF DESCRIPTION OF THE DRAWINGS

Preferred features of the present invention will now be described with reference to the accompanying drawings, where like reference numerals denote like parts, and in which:

FIG. 1 illustrates an exploded view of an expiratory valve unit according to the present invention;

FIG. 2 illustrates an exploded view of an alternative embodiment of an expiratory valve unit according to the present invention; and

FIG. 3 illustrates an exploded view of a respirator for use with the valve unit illustrated in FIG. 1 or FIG. 2.

### DETAILED DESCRIPTION OF THE PREFERRED EMBODIMENTS

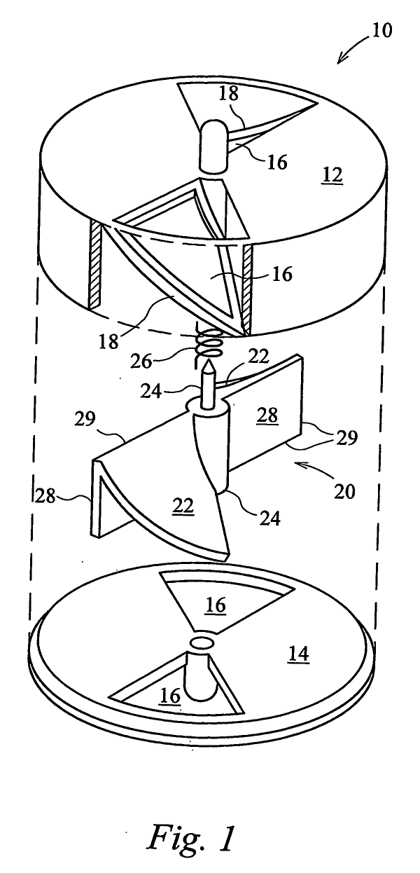

With reference to FIG. 1, an expiratory valve unit 10 comprises a body having upper 12 and lower 14 portions and a plurality of outlets 16 for user exhalate formed therein. In the illustrated embodiment, the body has two outlets 16, but additional outlets 16 may be provided. A valve seat 18 extends around each outlet 16 (a portion of the upper portion 14 is removed from FIG. 1 for clarity purposes only). A rotary closure device 20 is mounted within the body for rotation relative thereto. The closure member 20 includes a plurality of helical blades 22, one for each outlet 16, and has an axis of rotation 24 lying substantially parallel to the direction of exhalate flow through the outlets 16. The closure device is normally biassed by a spring 26 so that the blades 22 are forced against the valve seats 18 to close the outlets 16. In use, exhalate pressure exerted on the blades 22 resolves into axial and rotary forces, causing the closure member 20 to rotate against the force of the spring and open the outlets 16 to enable the exhalate to pass to the atmosphere. When the user has stopped exhaling, the spring 26 urges the blades back against the valve seats, thus closing the outlets 16. Vane-like surfaces 28 of the closure member, whose planes intersect the axis of rotation 24, provide damping by entering close fitting pockets between the upper and lower portions of the body. Air trapped in the pockets can escape only around the edge 29 of the surfaces 28, and thus prevent too rapid or unstable movement of the closure member when it opens and closes.

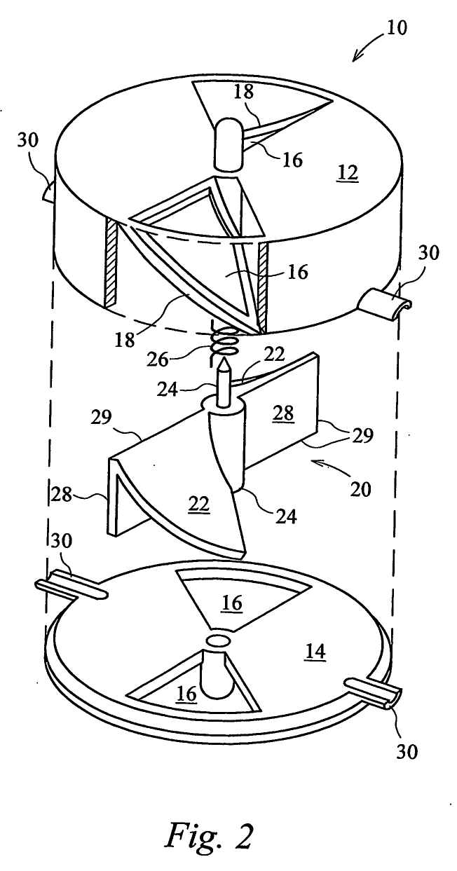

An alternative embodiment of the expiratory valve unit will now be described with reference to FIG. 2, where like reference numerals denote like parts. Normally the spring 26 holds the closure member closed against the pressure of the air supply to the user, and is set such that the user's exhalation pressure opens the valve against the spring. However any difference between the system pressure and the pressure in the damping pockets can disturb the correct operation of the valve. For example relatively low pressure in the pockets may result in the valve remaining open when it should be closed.

Therefore, in this embodiment the expiratory valve unit is provided with pressure compensation means, specifically in the form of small-bore spigots 30. A supply of air at system pressure is supplied via the spigots 30 to each of the damping chambers defined by the upper and lower portions 12, 14 of the unit 10 and the surfaces 28 of the closure member 20. The pressure in the damping chambers acting upon surfaces 28 of the closure member 20 counteracts, or balances, the rotary component of the force applied to the blades 22 by the system pressure. The pressure compensation means thus enables the torque exerted by spring 26 to be set accurately to respond only to the user's exhalation pressure. Because the spigots 30 are small-bore they apply the relatively constant supply pressure to the damping chambers but present a substantial impedance to the relatively sudden change in pressure when the user exhales. Thus a transient pressure increase due to exhalation is applied to the blades 22, but not immediately to the reverse side of the vanes 28 bounding the damping chambers. The valve thus opens under exhalation pressure and, when this subsides, is closed again by the spring.

The dimensions of the spigot 30 inlets, the elevated pressure air and the leakage paths around the surfaces 28 may be adjusted to provide differing compensation and damping characteristics.

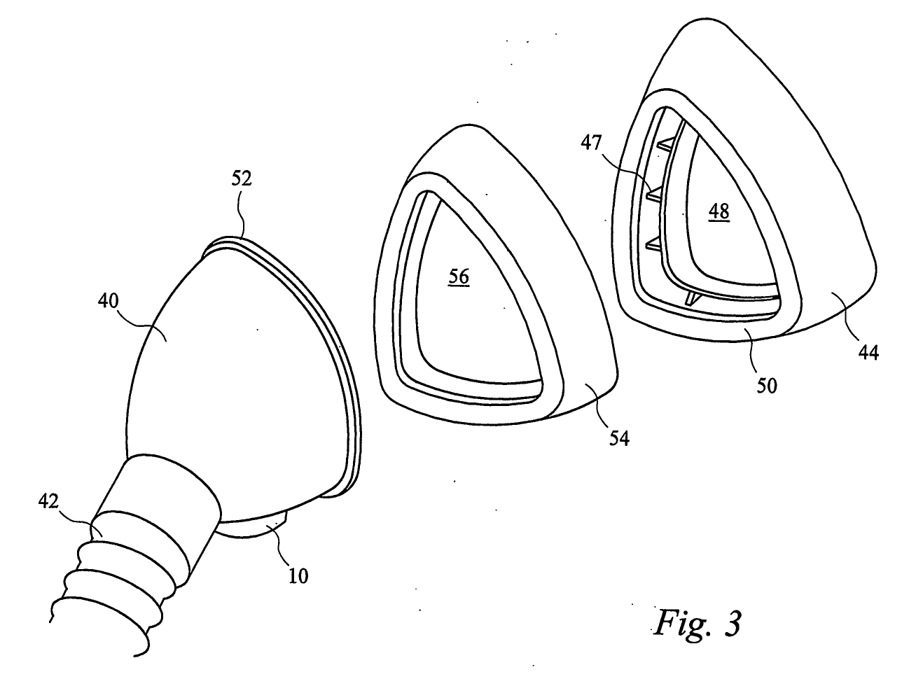

With reference to FIG. 3, the expiratory valve unit 10 of either the previously described embodiments is housed in a rigid unit 40 of a respirator. Unit 40 houses all of the common elements of the respirator, such as the remainder of the breathing apparatus, including an inspiratory valve unit, and a communications microphone. The unit 40 is connected to a supply hose 42 for the supply of breathing gas to a wearer, such as an airman. The unit 40 is a common element of the breathing mask, in that it is supplied in common to many airmen regardless of facial size and/or shape. The unit 40 is connected to a pre-formed unit 44 having a flexible body moulded from, for example, rubber material, for sealing to an airman's face. The inner surface of the body may be moulded with features 47 which prevent the reflex edge of the sealing surface of the unit from becoming inverted under pressure.

The pre-formed unit 44 is a sized component, which may also be shaped to suit differing facial characteristics, selected from a range of such units 44 according to the size and/or shape of the wearer's face. The units 40, 44 are assembled by threading the supply hose 42 though aperture 48 in the unit 44 and drawing the unit 44 around the unit 40 so that lip 50 of the unit 44 engages the raised edge 52 of the unit 40. A rigid clamping unit 54, which may be formed from moulded plastics material, is, like the unit 44, a sized component and selected from a range of similar units in accordance with the particular unit 44 chosen for the airman. The clamping unit 54 is assembled to the units 40, 44 by similarly threading the supply hose 42 through the aperture 56 and drawing the clamping unit 54 around unit 40 to engage the unit 44. The clamping unit 54 may be secured by a snap-fit or by any conventional fastening.

Each feature disclosed in the description, and/or the claims and drawings may be provided independently or in any appropriate combination. In particular a feature of a subsidiary claim may be incorporated in a claim from which it is not dependent.

The text of the abstract filed herewith is repeated here as part of the specification.

An expiratory valve unit comprises a body having a plurality of outlets and valve seats, and a rotary closure member having a plurality of blades each normally biassed against a respective seat to close the outlets and being rotatable relative to said body when under pressure from user exhalate impinging the blades to open the outlets.

1-19. (canceled)

20. An expiratory valve unit comprising a body having an outlet, and a rotary closure member mounted for rotation about an axis passing through the centre of gravity of the closure member, the closure member being normally biased against a valve seat to close the outlet and rotatable relative to said body when under pressure from user exhalate to open the outlet.

21. A valve unit according to claim 20, the body having a plurality of outlets, the closure member having a plurality of blades and being rotatable relative to said body when pressure from user exhalate acts on the blades to open the outlets.

22. An expiratory valve unit comprising a body having a plurality of outlets, and a rotary closure member having a plurality of blades, the closure member being normally biassed against a valve seat to close the outlets and rotatable relative to said body when pressure from user exhalate acts on the blades to open the outlets.

23. A valve unit according to claim 21, wherein the blades are symmetrically disposed about the closure member.

24. A valve unit according to claim 22, wherein the blades are symmetrically disposed about the closure member.

25. A valve unit according to claim 22, wherein each blade is helical.

26. A valve unit according to claim 22, wherein the axis of rotation of the closure member extends in the direction of exhalate flow through the outlets.

27. A valve unit according to claim 22, comprising a device for equalizing pressure differences across the closure member which would affect rotation thereof, other than pressure differences due to exhalate pressure.

28. A valve unit according to claim 22, comprising a damper for damping movement of the closure member.

29. A valve unit according to claim 28, wherein the damper comprises a part closely fitting a moveable in a closed chamber.

30. A valve unit according to claim 28, wherein the pressure equalizing device is arranged for equalizing the pressure between a user side of the unit and the closed chamber when the user is not exhaling.

31. A valve unit according to claim 29, wherein the pressure equalizing device comprises gas inlets on the body in communication with the closed chamber for providing gas at user side pressure thereto.

32. A respirator comprising a valve unit according to claim 20.

33. A respirator according to claim 32, comprising a first unit having a flexible body for forming a seal with a wearer's face, and, connected to the body, a second unit housing breathing apparatus including said valve unit.

34. A respirator according to claim 33, wherein the first unit comprises a device for preventing inversion of the sealing surface when under pressure.

35. A respirator according to claim 34, wherein said inversion prevention device is integral with said seal.

36. A respirator according to claim 33, comprising a rigid unit extending about the second unit for connecting the first unit to the second unit.

37. A respirator comprising a valve unit according to claim 22.

38. A respirator according to claim 37, comprising a first unit having a flexible body for forming a seal with a wearer's face, and, connected to the body, a second unit housing breathing apparatus including said valve unit.

39. A respirator according to claim 38, wherein the first unit comprises a device for preventing inversion of the sealing surface when under pressure.

40. A respirator according to claim 39, wherein said inversion prevention device is integral with said seal.

41. A respirator according to claim 38, comprising a rigid unit extending about the second unit for connecting the first unit to the second unit.
